(19) United States
(12) Patent Application Publication
(10) Pub. No.: US 2008/0317195 A1
(43) Pub. Date: Dec. 25, 2008

(54) MEDICAL-DIAGNOSIS ASSISTING APPARATUS, MEDICAL-DIAGNOSIS ASSISTING METHOD, AND RADIODIAGNOSIS APPARATUS

(75) Inventors: Hideaki KOBAYASHI, Otawara-shi (JP); Hitoshi YAMAGATA, Otawara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/137,144

(22) Filed: Jun. 11, 2008

(30) Foreign Application Priority Data
Jun. 20, 2007 (JP) 2007-162673
Apr. 10, 2008 (JP) 2008-102782

(51) Int. Cl. A61B 6/00

(52) U.S. Cl. 378/4; 382/132

(57) ABSTRACT

A medical-diagnosis assisting apparatus and a radiodiagnosis apparatus that include a simulation function prior to an examination/treatment, and a guide function during an examination/treatment are provided. Specifically, an extracting unit that extracts image data of a blood vessel portion to be an observation target from three-dimensional image data acquired by imaging a subject, a display unit that can display a three-dimensional image of the extracted blood vessel portion, a display-direction setting unit that displays the three-dimensional image of the extracted blood vessel portion on a display unit at a display angle specified by a user, and a simulation-image creating unit that simulates a course of a catheter when the catheter is to be inserted into the extracted blood vessel portion, and overlays a maker that indicates a position and a moving direction of the catheter on the three-dimensional image of the blood vessel portion are included.

### CROSS-REFERENCE TO RELATED APPLICATIONS

This application is based upon and claims the benefit of priority from the prior Japanese Patent Application No. 2007-162673, filed on Jun. 20, 2007; and Japanese Patent Application No. 2008-102782, filed on Apr. 10, 2008, the entire contents of which are incorporated herein by reference.

### BACKGROUND OF THE INVENTION

1. Field of the Invention

The present invention relates to a medical-diagnosis assisting apparatus and a medical-diagnosis assisting method that assist an operator and improve safety during a medical diagnosis by displaying a simulation image or a guide image particularly when performing an examination or a treatment for a disease by inserting a catheter into a subject. The present invention also relates to a radiodiagnosis apparatus, such as an X-ray CT apparatus and an X-ray circulatory-diagnosis apparatus to which the medical-diagnosis assisting apparatus is applied.

2. Description of the Related Art

Conventionally, a medial system that uses an X-ray CT apparatus and an X-ray circulatory-diagnosis apparatus performs diagnostic imaging with a computer, carries out a diagnosis while referring to image data imaged from a subject, and assists an examination or a treatment for an operator.

For example, to perform a diagnosis of ischemic heart disease, an examination or a treatment is performed by using a catheter, which is less invasive than a surgical operation. To perform a catheter examination/treatment, for example, a catheter is inserted into a coronary artery of a subject and a contrast medium is injected, and then a lesion portion (a stenotic portion of a blood vessel etc.) is searched while a two-dimensional perspective image of the coronary artery acquired by irradiating an X-ray to the subject is being watched.

A stent is then made to stay at the searched lesion portion, and a treatment to expand the stenotic portion is given. The stent is inserted into the stenotic portion of the blood vessel, and a diameter of the blood vessel is maintained by expanding the stent by using the catheter with a balloon. In addition to when a search for a lesion portion or a treatment is made, the above examination is also performed when observing conditions and confirming a progress of a lesion portion after a treatment, so that such examination is generally performed a plurality of number of times.

However, despite a common belief that a catheter examination/treatment is less invasive compared with a surgical operation, there is a possibility of developing a complication due to a catheter operation or a stay of a stent (expansion operation), so that it is desired to reduce the number of times of examinations to as few times as possible.

Moreover, during an examination/treatment, a two-dimensional perspective image is acquired by irradiating X-rays while injecting a contrast medium, and a diagnosis is performed while the image is being watched. However, sufficient information cannot be obtained in some cases depending on an irradiation angle, so that a perspective image needs to be reacquired by changing an irradiation direction as required. Consequently, an examination time tends to be long, and a burden onto a patient (subject) due to a contrast medium injection and an X-ray exposure is increased.

In relation to an angiography with administration of a contrast medium, a blood-vessel extraction algorithm to be used by an X-ray CT apparatus to extract only a coronary artery of a contrasted blood-vessel region from a 3D volume image is described in the following document 1:

O. Wink, W. J. Niessen, M. A. Viergever, “Fast Delineation and Visualization of Vessels in 3-D Angiographic Images”, IEEE Trans. Med. Imaging, Vol. 19, No. 4, p. 337-346, April 2000.

Conventionally, there is a possibility of developing a complication caused by a catheter operation or a stay of a stent (expansion operation), so that it is desired to reduce the number of times of examinations to as few times as possible. Moreover, during an examination/treatment, a two-dimensional perspective image is acquired by irradiating X-rays while injecting a contrast medium, however, sometimes sufficient information cannot be obtained in some cases depending on an angle, so that a perspective image needs to be reacquired by changing an irradiation direction as required. Consequently, an examination time tends to be long, and a burden onto a patient (subject) is increased.

### SUMMARY OF THE INVENTION

According to one aspect of the present invention, a medical-diagnosis assisting apparatus includes an extracting unit that extracts image data of a blood vessel portion to be an observation target from three-dimensional image data acquired by imaging a subject; a display unit that can display a rendering image of the blood vessel portion extracted by the extracting unit; a display-direction setting unit that displays the rendering image of the extracted blood vessel portion on the display unit at a display angle specified by a user; and a simulation-image creating unit that simulates a course of a catheter when the catheter is inserted into the blood vessel portion extracted by the extracting unit, and overlays a maker indicating a position and a moving direction of the catheter on the rendering image of the blood vessel portion.

According to another aspect of the present invention, a radiodiagnosis apparatus includes a projection-image data creating unit that creates perspective image data of a subject with an X-ray diagnostic apparatus; an extracting unit that acquires three-dimensional image data obtained by imaging the subject with any one of an X-ray CT apparatus and a magnetic-resonance diagnostic apparatus, and extracts image data of a blood vessel portion to be an observation target; a display-direction setting unit that sets a display angle of a rendering image of the blood vessel portion extracted by the extracting unit based on a specification specified by a user; and a display unit that can display a perspective image of the blood vessel portion created by the projection-image data creating unit, and a rendering image of the blood vessel portion at the display angle specified by the user.

According to still another aspect of the present invention, a medical-diagnosis assisting method includes extracting image data of a blood vessel portion to be an observation target from three-dimensional image data acquired by imaging a subject; creating a maker that indicates a position and a moving direction of a catheter by simulating a course of the catheter when the catheter is inserted into the extracted blood vessel portion; displaying a three-dimensional image of the extracted blood vessel portion on a display unit at a display angle specified by a user; and displaying the maker by overlaying on the three-dimensional image of the blood vessel portion.

### BRIEF DESCRIPTION OF THE DRAWINGS

FIG. 1 is a schematic diagram illustrating a system configuration of a medical system to which a medical-diagnosis assisting apparatus according to an embodiment of the present invention is applied;

FIG. 2 is a block diagram illustrating an example of an X-ray CT apparatus as a radiodiagnosis apparatus according to an embodiment of the present invention;

FIG. 3 is a block diagram illustrating an example of an X-ray circulatory-diagnosis apparatus as a radiodiagnosis apparatus according to an embodiment of the present invention;

FIG. 4 is a block diagram illustrating a medical-diagnosis assisting apparatus according to a first embodiment of the present invention;

FIG. 5 is a flowchart for explaining operation according to the first embodiment;

FIGS. 6A and 6B are schematic diagrams for explaining a GUI screen for setting parameters according to the first embodiment;

FIGS. 7A to 7C are schematic diagrams for explaining a renewal determination method of a position and a direction of a catheter according to the first embodiment;

FIGS. 8A to 8C are schematic diagrams for explaining creation and expansion of segment data according to the first embodiment;

FIGS. 9A and 9B are schematic diagrams for explaining a simulation image on a display unit according to the first embodiment;

FIGS. 10A and 10B are schematic diagrams for briefly explaining movement of the X-ray circulatory-diagnosis apparatus as a radiodiagnosis apparatus according to the embodiments of the present invention;

FIG. 11 is a flowchart for explaining operation of a radiodiagnosis apparatus according to a second embodiment of the present invention;

FIGS. 12A and 12B are schematic diagrams for explaining a guide image on a display unit according to the second embodiment of the present invention;

FIG. 13 is a block diagram illustrating a configuration of a medical-diagnosis assisting apparatus according to a third embodiment of the present invention;

FIG. 14 is a schematic diagram illustrating a change in visualization due to an injection of a dose of a contrast medium correspondingly to an electrocardiographic wave and coronary-artery flow-rate change;

FIG. 15 is a schematic diagram for explaining a GUI screen for setting parameters according to the third embodiment;

FIG. 16 is a schematic diagram illustrating a setting of starting/ending positions on image data in all time phases; and

FIG. 17 is a flowchart for explaining operation of the medical-diagnosis assisting apparatus according to the third embodiment.

### DETAILED DESCRIPTION OF THE INVENTION

Three embodiments of the present invention will be explained below in detail with reference to the drawings.

### First Embodiment

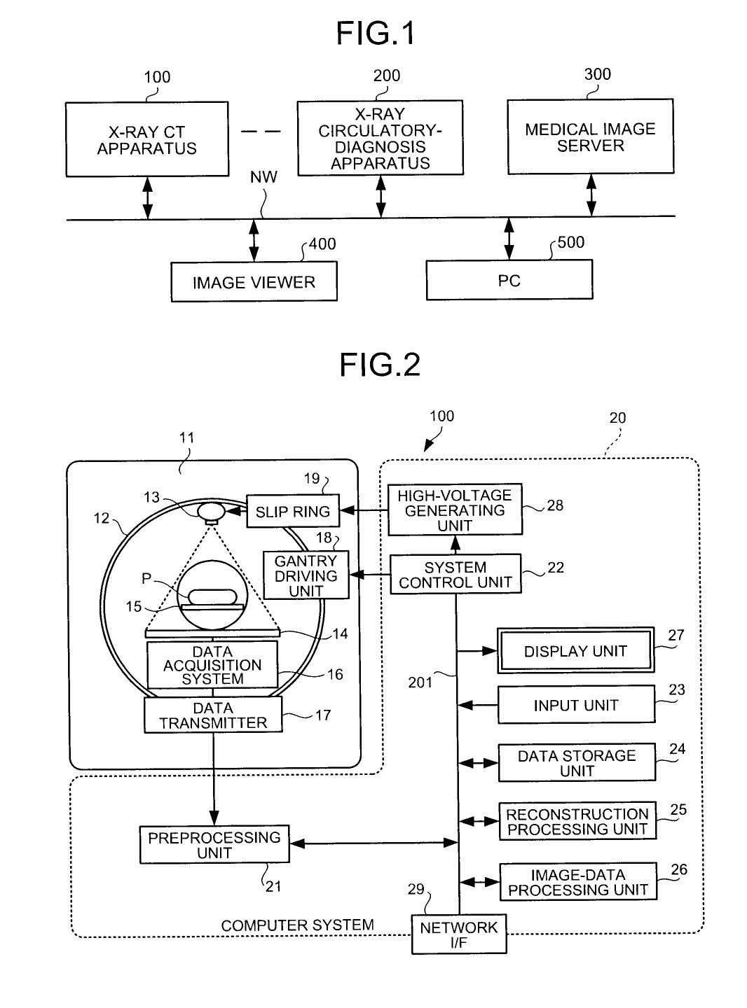

FIG. 1 is a schematic diagram illustrating a system configuration of a medical system to which a medical-diagnosis assisting apparatus according to an embodiment of the present invention is applied. The medical system shown in FIG. 1 includes modalities of radiodiagnosis apparatuses, such as an X-ray CT apparatus 100, and an X-ray circulatory-diagnosis apparatus 200. The modalities are connected to a network NW, and the network NW is connected to a medical image server 300 that stores therein medical image information (including image data and additional information). The network NW is connected to an image viewer terminal 400 and an input-output terminal 500.

The X-ray CT apparatus 100 and the X-ray circulatory-diagnosis apparatus 200 are configured to image a subject and generate image data. The image data are stored in the medical image server 300. The image viewer terminal 400 acquires and processes image data and patient information stored in the medical image server 300, and then displays various information. The input-output terminal 500 is a PC (Personal Computer) that inputs and outputs information into and from the system by logging in various devices on the network NW.

In the system shown in FIG. 1, a doctor gives, for example, an order for a radiographic examination via the input-output terminal 500, and an engineer performs the examination based on the order by operating the X-ray CT apparatus 100 and the X-ray circulatory-diagnosis apparatus 200. Medical image data imaged by a modality, such as the X-ray CT apparatus 100, are stored in the medical image server 300.

Moreover, the medical image data are added with additional information, such as a patient ID, a patient name, age, sexuality, an examined portion, and the like, and stored in the medical image server 300 so that various searches can be performed based on the additional information. The image viewer terminal 400 further displays on a display unit various information, such as image data, in accordance with, for example, processing of creating a medical image list or a patient list, or a request from a user (a doctor, an engineer, an operator, or the like). Moreover, the image viewer terminal 400 has a simulation function and a guide function, and is configured to display on the display unit an image for assisting the user when the user performs an examination or a treatment.

FIG. 2 is a schematic diagram illustrating a configuration of an embodiment of the X-ray CT apparatus 100 as a radiodiagnosis apparatus. In FIG. 2, the X-ray CT apparatus 100 includes a gantry 11, inside which a rotational ring 12 is provided, and rotated by a rotating mechanism, which is not shown. Inside the rotational ring 12, an X-ray tube 13 that generates X-rays to a subject P placed in an effective field of view is attached.

Additionally, a radiation detector 14 is arranged opposite to the X-ray tube 13, a center area of the rotational ring 12 is opened, and the subject P placed on a patient couch top 15 is inserted into the center area. An X-ray passed through the subject P and detected by the radiation detector 14 is converted into an electric signal, amplified by a data acquisition system (DAS) 16, and converted into digital data. The X-ray tube 13 and the radiation detector 14 constitute an imaging unit.

The radiation detector 14 includes a plurality of detector modules. Each of the detector modules includes a plurality of sensor arrays that includes a scintillator array and a photodiode array, and the detector modules are arranged along an arc with the center on a focal point of the X-ray tube 13.

Digital data (projection data) from the DAS 16 are transmitted to a computer system 20 via a data transmitter 17. The gantry 11 is further provided with a gantry driving unit 18 and a slip ring 19.

The computer system 20 is provided on a console. The projection data from the data transmitter 17 are supplied to a preprocessing unit 21. The preprocessing unit 21 performs preprocessing, such as data correction, on the projection data, and outputs preprocessed data to a bus line 201.

The bus line 201 is connected to a system control unit 22, an input unit 23, a data storage unit 24, a reconstruction processing unit 25, an image-data processing unit 26, a display unit 27, and the like.

The system control unit 22 functions as a host controller, and controls operations of each unit of the computer system 20, and the gantry driving unit 18 and a high-voltage generating unit 28. The data storage unit 24 stores therein data, such as tomographic images. The reconstruction processing unit 25 reconstructs three-dimensional (3D) image data from projection data. The image-data processing unit 26 processes data stored in the data storage unit 24, or reconstructed image data. The display unit 27 displays thereon information, such as images obtained by image data processing.

The input unit 23 includes a keyboard, a mouse, and the like, is operated by the user, and receives various settings for data processing. Moreover, the input unit 23 receives input of various information, such as conditions of a patient, and an examination method.

The high-voltage generating unit 28 supplies electric power to the X-ray tube 13 via the slip ring 19, and provides electric power (a tube voltage and a tube current) required for irradiation of X-rays. The X-ray tube 13 generates beam X-rays that extend in two directions, namely, a slice direction and a channel direction. The slice direction is in parallel with the body axis direction of the subject P, and the channel direction is orthogonal to the slice direction.

Moreover, the bus line 201 is provided with a network interface 29, and the X-ray CT apparatus 100 is configured connectable to the network NW (FIG. 1), so that imaged image data and reconstructed image data by the X-ray CT apparatus 100 are stored into the medical image server 300.

The X-ray CT apparatus 100 sets a scan range and performs a volume scan (3D scan), and reconstructs a 3D (three dimensional) image with the reconstruction processing unit 25, thereby acquiring the 3D image within the scan range.

Additionally, to observe organs, such as blood vessels, the X-ray CT apparatus 100 images a subject by administrating a contrast medium to the subject in some cases. When imaging blood vessels with administration of a contrast medium, X-ray CT image data of contrasted blood vessels are reconstructed, and 3D image data are created from the X-ray CT image data.

A method of creating 3D image data can be, for example, Maximum Intensity Projection (MIP), Minimum Intensity Projection, or average projection (X-ray Projection). MIP is a method of displaying a maximum value in a projection route from among results of projection processing performed in an arbitrary direction, and Minimum Intensity Projection is a method of projecting a minimum value.

A 3D image (MIP image) created by MIP is often used for observation of contrasted blood vessels. Moreover, VR (Volume Rendering) or SVR (Shaded Volume Rendering), each of which is a method of reconstructing and visualizing a stereoscopic image by using pixel values (CT values) and opacity, are used. SVR is suitable for a dynamic observation. For example, a moving image of heart wall motion can be displayed with shade.

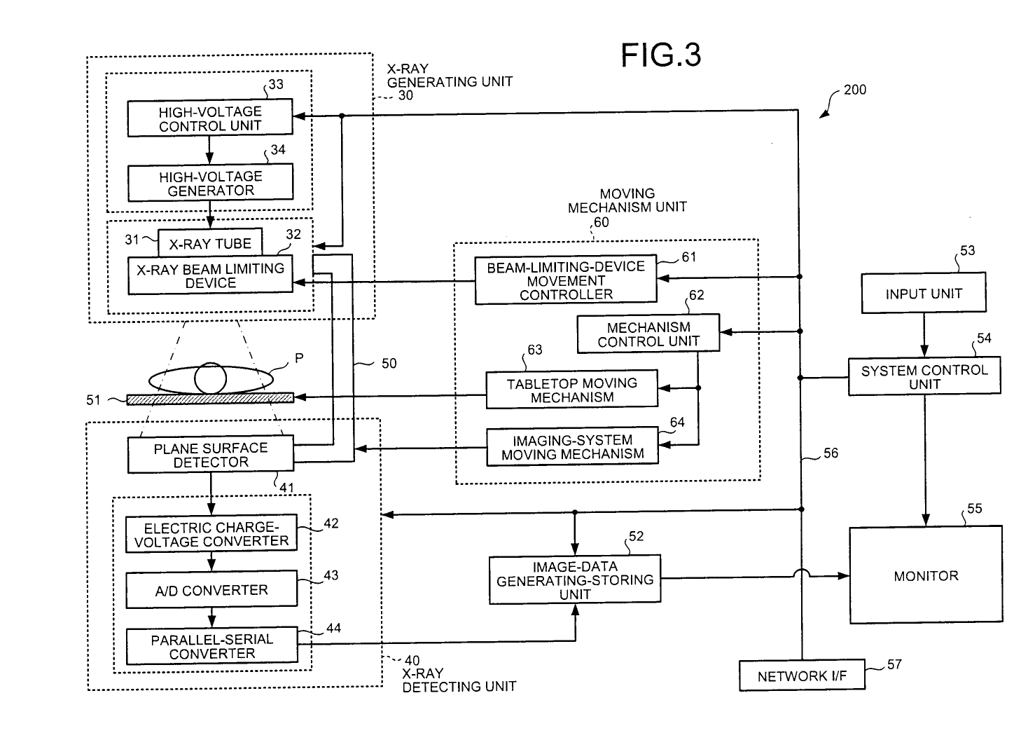

FIG. 3 is a schematic diagram illustrating a configuration of the X-ray circulatory-diagnosis apparatus 200 as a radiodiagnosis apparatus according to an embodiment of the present invention. According to FIG. 3, the X-ray circulatory-diagnosis apparatus 200 includes an X-ray generating unit 30 configured to generate X-rays to the subject P, and an X-ray detecting unit 40 that detects X-rays passed through the subject P two-dimensionally, and creates X-ray projection data based on a detection result.

The X-ray generating unit 30 includes an X-ray irradiating unit that includes an X-ray tube 31 and an X-ray beam limiting device 32, and a high-voltage generating unit that includes a high-voltage control unit 33 and a high-voltage generator 34. The X-ray tube 31 is a vacuum tube that generates X-rays, and generates an X-ray by accelerating electrons emitted from the cathode (filament) with a high voltage and colliding the electrons to a tungsten anode.

The X-ray detecting unit 40 includes a plane surface detector 41, an electric charge-voltage converter 42, an A/D converter 43, and a parallel-serial converter 44. The electric charge-voltage converter 42 converts electric charges read from the plane surface detector 41 to voltages. The A/D converter 43 converts output of the electric charge-voltage converter 42 to digital signals. The parallel-serial converter 44 converts X-ray projection data that are read and digitized per line in parallel by the plane surface detector 41 into a time series signal.

The X-ray generating unit 30 and the X-ray detecting unit 40 are supported by an arm (C-arm) 50. The arm 50 can move, for example, in the body axis direction of the subject P, and can rotate around the subject P. The X-ray generating unit 30 and the X-ray detecting unit 40 constitute an imaging unit.

The X-ray circulatory-diagnosis apparatus 200 further includes a moving mechanism unit 60. The moving mechanism unit 60 includes a beam-limiting-device movement controller 61 and a mechanism control unit 62. The beam-limiting-device movement controller 61 controls movement of aperture blades and other parts in the X-ray beam limiting device 32. The mechanism control unit 62 controls movements of a moving mechanism 63 of a tabletop 51 on which the subject P is placed, and a imaging-system moving mechanism 64.

The X-ray circulatory-diagnosis apparatus 200 further includes an image-data generating-storing unit 52, an input unit 53, a system control unit 54, and a monitor 55. The image-data generating-storing unit 52 generates and stores therein perspective image data based on X-ray perspective image data from the parallel-serial converter 44, and the monitor 55 displays thereon the perspective image data created by the image-data generating-storing unit 52.

The input unit 53 is for the user, such as a doctor, to input various commands and other information to the X-ray circulatory-diagnosis apparatus 200, and includes interactive interfaces including input devices, such as a mouse, a keyboard, a trackball, and a joystick, and a display panel or various switches. The system control unit 54 totally controls each unit of the X-ray circulatory-diagnosis apparatus 200 via a bus line 56.

The bus line 56 is connected to a network interface 57, and the X-ray circulatory-diagnosis apparatus 200 is configured connectable to the network NW (FIG. 1), so that image data imaged by the X-ray circulatory-diagnosis apparatus 200 can be stored in the medical image server 300.

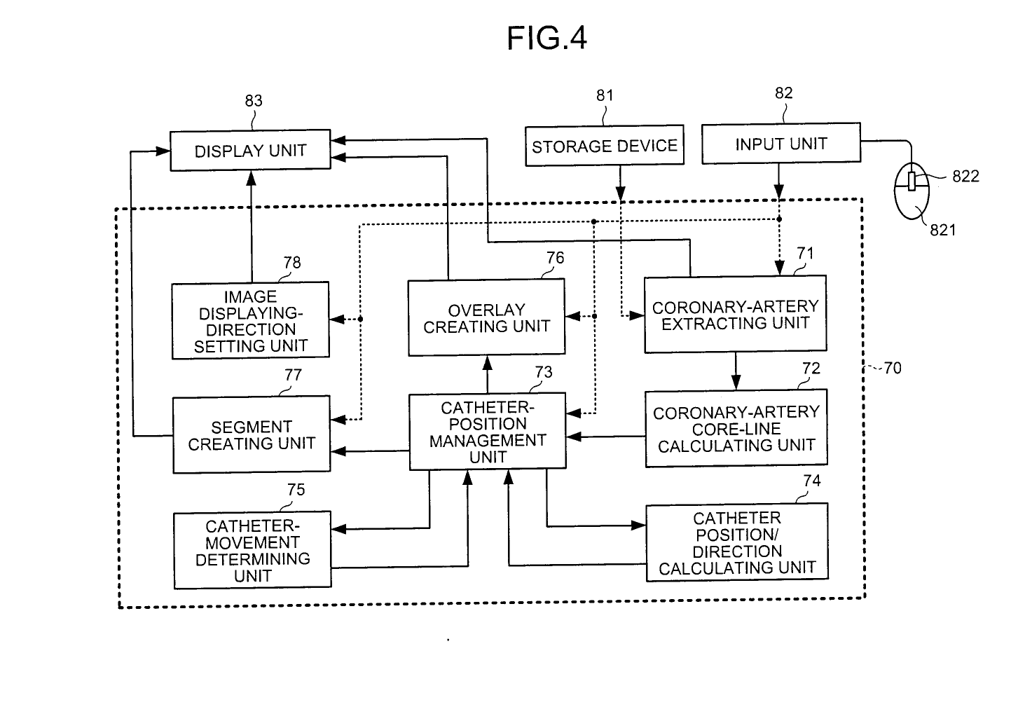

A configuration and functions of principal parts of the embodiments according to the present invention are explained below. FIG. 4 is a block diagram illustrating a medical-diagnosis assisting apparatus 70 that has a simulation function. The medical-diagnosis assisting apparatus 70 is provided, for example, inside the image viewer terminal 400, and configured to perform a simulation before a catheter examination/treatment.

According to FIG. 4, 71 denotes a coronary-artery extracting unit, which reads three-dimensional (3D) image data stored in a storage device 81, and extracts a coronary artery (LCA: left coronary artery, RCA: right coronary artery) based on CT values of the read image data. The storage device 81 stores therein three-dimensional image data that are collected and reconstructed by the X-ray CT apparatus 100, and can be the medical image server 300 in FIG. 1.

The coronary-artery extracting unit 71 is connected to a coronary-artery core-line calculating unit 72. The coronary-artery core-line calculating unit 72 calculates a core line of a coronary artery extracted by the coronary-artery extracting unit 71. The coronary-artery core-line calculating unit 72 is further connected to a catheter-position management unit 73, and the catheter-position management unit 73 is connected to a catheter position/direction calculating unit 74 and a catheter-movement determining unit 75.

The catheter-position management unit 73 determines a current position of a catheter and a direction to which the catheter is to be moved based on information provided from an input unit 82, the coronary-artery core-line calculating unit 72, the catheter position/direction calculating unit 74, and the catheter-movement determining unit 75. The input unit 82 includes, for example, a mouse and a keyboard, and is operated by the user (doctor, engineer, or the like). The figure depicts an example that the input unit 82 includes a mouse 821.

When a renewal of a catheter position/direction is requested from the input unit 82, the catheter-position management unit 73 provides information for determination to the catheter-movement determining unit 75, and acquires information whether the catheter position/direction can be renewed from the catheter-movement determining unit 75.

Moreover, when the catheter-movement determining unit 75 determines that the catheter position/direction can be renewed, or when the catheter position/direction is in the initial state in which it has not been set yet, the catheter-position management unit 73 provides information for calculation to the catheter position/direction calculating unit 74, and acquires information about a current catheter position/direction from the catheter position/direction calculating unit 74.

The catheter position/direction calculating unit 74 calculates a position and a direction of the catheter based on the information provided by the catheter-position management unit 73. The catheter-movement determining unit 75 determines whether the catheter position/direction can be renewed (moved), i.e., whether the catheter can go ahead, based on the information provided by the catheter-position management unit 73.

Furthermore, the catheter-position management unit 73 is connected to an overlay creating unit 76. The overlay creating unit 76 creates an overlay image to be displayed on a three-dimensional image of a coronary artery. An image created by IP (Intensity Projection) or SVR (Shaded Volume Rendering) is used as the three-dimensional image, and an arrow (marker) image that indicates a position and a direction of the catheter is created as the overlay image. Data of the three-dimensional image and the overlay image are supplied to and displayed on a display unit 83.

Thus, the coronary-artery core-line calculating unit 72, the catheter-position management unit 73, the catheter position/direction calculating unit 74, the catheter-movement determining unit 75, and the overlay creating unit 76 constitute a simulation-image creating unit.

The medical-diagnosis assisting apparatus 70 is further provided with a segment creating unit 77 and an image displaying-direction setting unit 78. The segment creating unit 77 creates segment data to be displayed on a three-dimensional image of a coronary artery based on information provided from the input unit 82 and information from the catheter-position management unit 73. The image displaying-direction setting unit 78 changes a display angle of a three-dimensional image of a coronary artery by controlling the display unit 83.

The medical-diagnosis assisting apparatus 70 can display to the user a three-dimensional image of a coronary artery viewed from an arbitrary angle by using image data imaged by the X-ray CT apparatus 100 in advance. Moreover, the medical-diagnosis assisting apparatus 70 performs a simulation of an insertion of a catheter prior to an examination or a treatment.

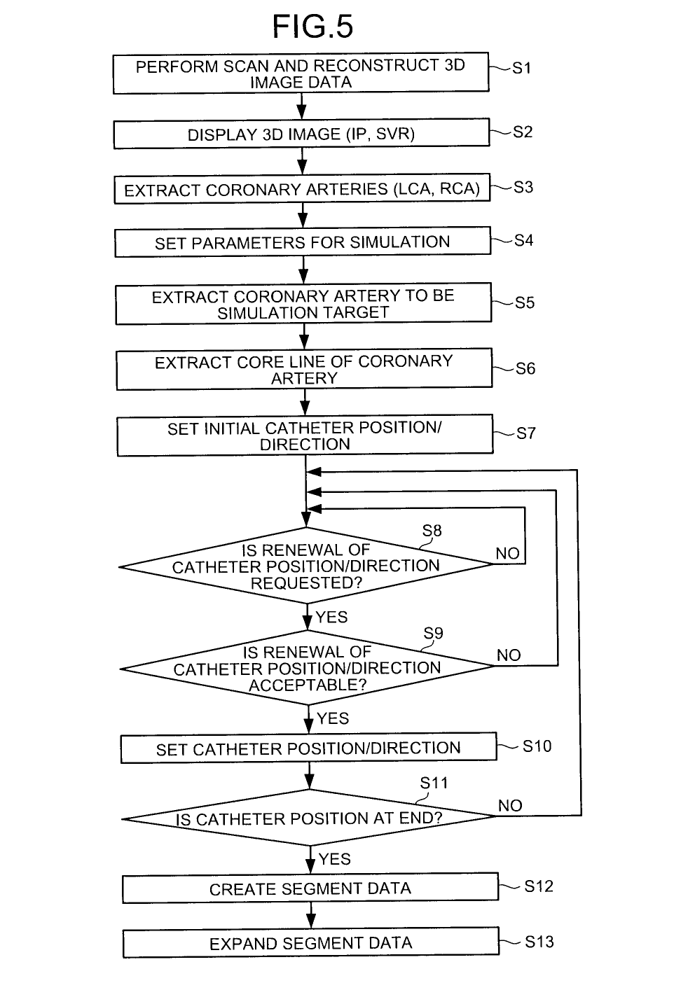

Operation of the medical-diagnosis assisting apparatus 70 is explained below with reference to a flowchart shown in FIG. 5. FIG. 5 describes a procedure of performing a simulation of a catheter examination by extracting a coronary artery from X-ray CT image data and using the extracted image of the coronary artery.

A simulation image represents a state of the catheter in progress in accordance with a situation inside the blood vessel. The catheter is moved to a lesion portion (for example, a stenotic portion of the blood vessel) by turning the tip of the catheter, and when the catheter reaches the lesion portion, a segment (stent) is displayed.

First of all, at Step S1 in FIG. 5, to collect contrasted blood-vessel images of a subject, the X-ray CT apparatus 100 performs a scan with administration of a contrast medium, and reconstructs three-dimensional image data of the subject. The reconstructed three-dimensional image data are stored in the storage device 81.

At Step S2, the three-dimensional image data stored in the storage device 81 are read, and three-dimensional images (a 3D projection image and a 3D volume image) are displayed on the display unit 83. The 3D projection image can be displayed by switching Maximum Intensity Projection, Minimum Intensity Projection, and X-ray Projection. The projection methods are referred to as IP (Intensity Projection) in total. On the other hand, as the 3D volume image, a shaded volume rendering image (SVR: Shaded Volume Rendering) is displayed.

The displayed 3D projection image and the displayed 3D volume image include contrasted blood vessels and regions to be deleted for the simulation. The 3D projection image presents a region displayed on the 3D volume image as a projection image. Consequently, when segment processing, such as a region extraction, is executed on the 3D volume image, display of the 3D projection image is inevitably renewed.

Then at Step S3, only coronary arteries (LCA: left coronary artery, RCA: right coronary artery) that are a contrasted blood-vessel region are extracted from the 3D volume image based on CT values of the image data. As a blood-vessel extraction algorithm, for example, a method described in the aforementioned document 1 is used.

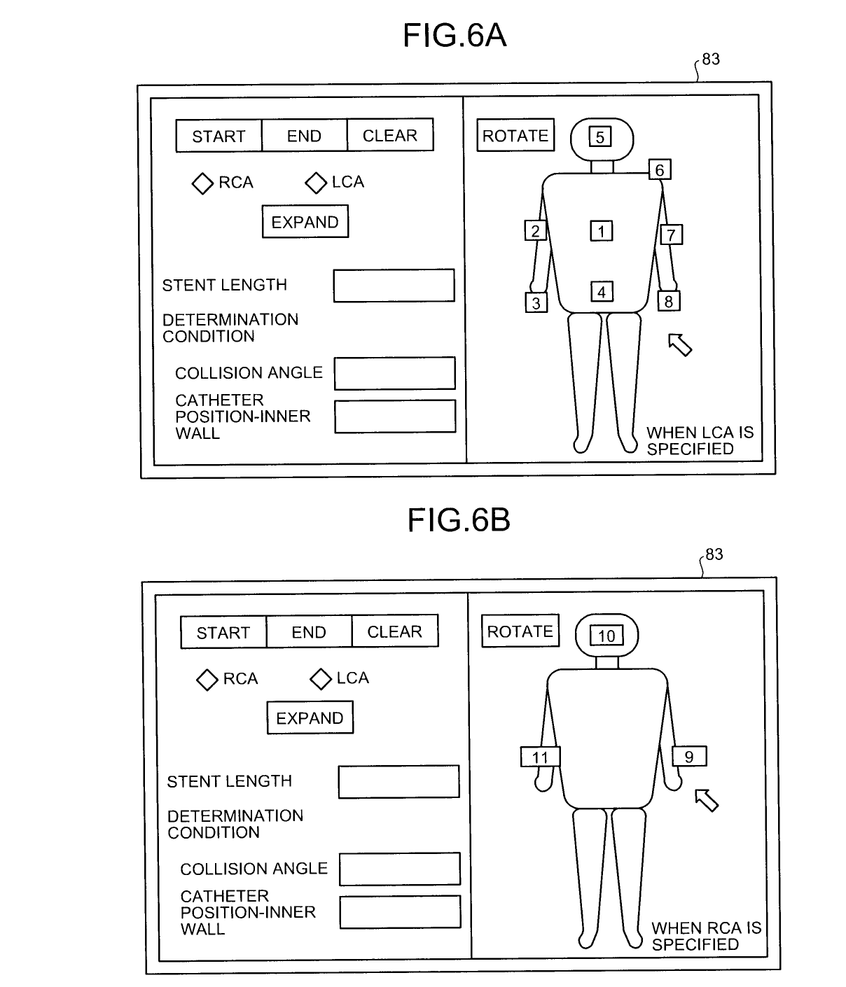

At Step S4, parameters to be required for performing the simulation are set. An example of a GUI (graphical user interface) for setting the parameters is shown in FIG. 6A. The GUI is an operational screen displayed on the screen to be used for setting the parameters. As shown on the left screen in FIG. 6A, the parameters to be set are, for example, items (a) to (e) as follows.

(a) Selection of a coronary artery: a coronary artery (LCA or RCA) as a simulation target is specified.

(b) Starting/ending positions: a range to be simulated (starting position and ending position) is specified.

(c) Determination condition (collision angle): an angle between the catheter tip and a blood-vessel inner wall is specified as a condition when determining whether the catheter can be moved during the simulation.

(d) Determination condition (catheter position—inner wall): a distance between the catheter and a blood-vessel inner wall is specified as a condition when determining whether the catheter can be moved during the simulation.

(e) Stent length: a length of the stent to stay at a lesion portion (a stenotic portion) is set.

Then at Step S5, as the coronary artery (LCA or RCA) that is a simulation target is specified at Step S4 (in the item a), only the specified coronary artery is extracted from the 3D volume image.

At Step S6, a core line of the extracted coronary artery is extracted within the simulation range specified at Step S4 (in the item b). The extracted core line is to be a movement course of the catheter during the simulation. As a core-line extraction algorithm, for example, a method described in the aforementioned document 1 is used.

At Step S7, initial catheter position and direction are set by using the simulation range (only the starting position at this step) specified at Step S4 (in the item b) similarly to Step S6.

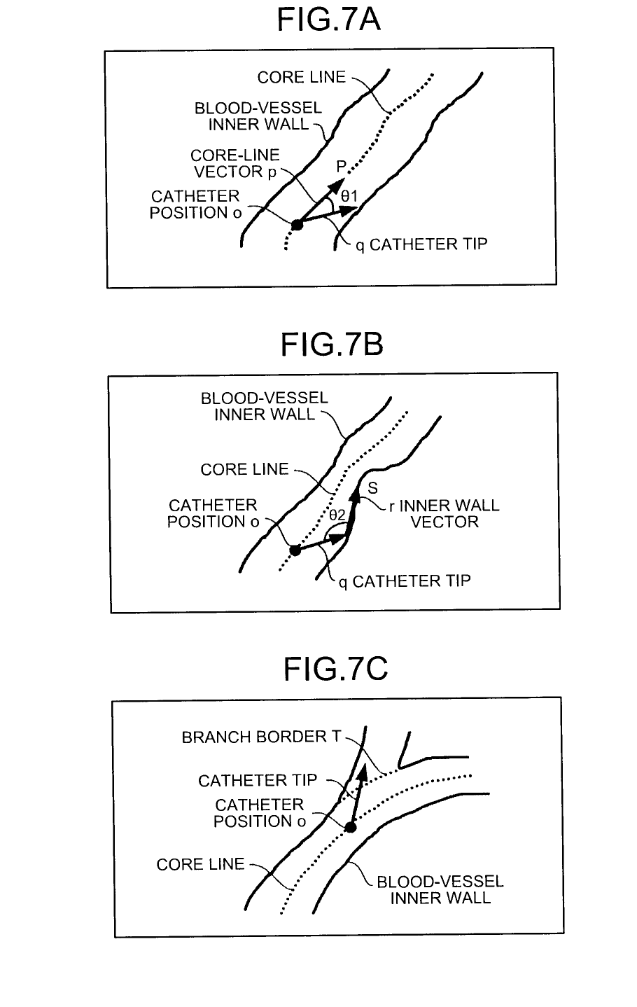

FIG. 7A is a schematic diagram that depicts the position and the direction of a catheter in a blood vessel, and the initial catheter position is a starting position o specified at Step S4 (in the item b), which is positioned on a core line (dotted line). The catheter direction is defined by a vector q that is represented by a certain angle θ1 from a core-line vector p with respect to the catheter position o as a base point, and means the catheter tip.

In other words, the catheter position o is determined on the core line (dotted line) based on input information. A point P on the core line at a distance of a few centimeters from the catheter position in a straight line is then obtained, and the core-line vector p is calculated. The point P on the core line is searched from the catheter position o in a direction of a simulation ending position.

The vector q, which is slanted from the core-line vector p by the angle θ1 with respect to the catheter position o is calculated. Consequently, the vector q that is calculated is assumed as the catheter tip. The angle θ1 can be arbitrarily changed by setting an environment for the simulation.

After the catheter position/direction is set, then at Step S8, the medical-diagnosis assisting apparatus 70 is in a stand-by state of waiting a new input (a renewal request for the catheter position/direction) from the user.

If a renewal is requested at Step S8, it is determined at Step S9 whether the renewal is acceptable based on input information from a mouse of the input unit 82. There are two conditions for the determination, and values set at Step S4 (in the items c and d) are used.

Determination on the collision angle set at the item c is performed first. The collision angle is, as shown in FIG. 7B, represented by the angle θ2 between the vector q of the catheter tip and a tangent vector r of the inner wall of the coronary artery, and if the angle θ2 between the two vectors is acuter than a predetermined angle, it is determined that the catheter cannot be moved. Because there is a possibility that the catheter tip at an acute angle may damage the blood-vessel inner wall, movement of the catheter is unacceptable.

In other words, a point S on the inner wall at a distance of a few millimeters from a position at which the catheter tip is in contact with the blood-vessel inner wall is obtained, and then the angle θ2 is calculated. The point S on the inner wall is searched toward a direction of the simulation ending position from the position at which the catheter tip is in contact with the inner wall.

If the angle θ2 that is calculated is acuter than the predetermined angle, it is determined that a renewal of the catheter position/direction is unacceptable. The distance from the position at which the catheter tip is in contact with the inner wall to the point S on the inner wall, and the angle θ2 can be arbitrarily changed by setting an environment of the simulation.

Determination on the “catheter position—inner wall” set at the item d is then performed. As shown in FIG. 7C, even when a vector of the catheter tip that is set from the catheter position o positioned on the core line of the coronary artery is an obtuse angle, if a distance from the catheter tip to a contact with an inner wall of the coronary artery is longer than a predetermined distance, the “catheter position—inner wall” is determined that movement of the catheter is unacceptable. In other words, the catheter is prevented from going into a branch from the main stream of the blood vessel along which the catheter is to be moved.

This means that a distance from the catheter position o to a contact with a blood-vessel inner wall is calculated. As a result, if the calculated distance to the inner wall is longer than the predetermined length, it is determined that a renewal of the catheter position/direction is unacceptable. The predetermined value defined as the catheter position—the inner wall is used for determination of a branch point as shown in FIG. 7C, it is desirable that the predetermined value is to be by taking into account a branch border T (an inner wall when the blood vessel does not branch).

If any of the two conditions is met, the renewal request for the catheter position is discarded, and the process control goes back to Step S8.

Determination processing at Step S9 is to be performed only if the input information from the input unit 82 is a “catheter-position renewal request”, and if it is a “catheter-direction renewal request”, the catheter direction is to be renewed unconditionally.

The input unit 82 includes, for example, the mouse 821 (see FIG. 4), and input information from the input unit 82 is assumed to include three kinds of information, namely, forward movement and backward movement of the catheter position, and turning movement of the catheter tip direction. For example, the catheter position is moved forward by one step by clicking the left button of the mouse 821, and continuously moved forward by pressing it continuously. Moreover, the catheter position is moved backward by one step by clicking the right button of the mouse 821, and continuously moved backward by pressing it continuously.

A wheel 822 can give an instruction of a direction of the catheter tip, the catheter tip is turned to the left with respect to the core line by turning the wheel 822 upward, and the catheter tip is turned to the right with respect to the core line by turning the wheel 822 downward.

Not limited to the mouse, for example, a GUI via which the user can specify an amount of movement and a turning angle can be used.

If it is determined that a “renewal of the catheter position/direction is acceptable” at Step S9, a catheter position/direction after the renewal is set at Step S10 based on the current catheter position/direction and the input information.

After the catheter position/direction is renewed at Step S10, it is determined at Step S11 whether the catheter position has reached the ending position set in the item b at Step S4. If the catheter position has reached the ending position, any further renewal request for the catheter position/direction is received, and the process control goes to Step S12. If the catheter position has not reached the ending position, the process control goes back to Step S8, and waits a new renewal request.

At Step S12, as the catheter position has reached the ending position, assuming that the stent is placed at a lesion portion (a stenotic portion of the blood vessel), segment data X is created by using the stent length set in the item e at Step S4.

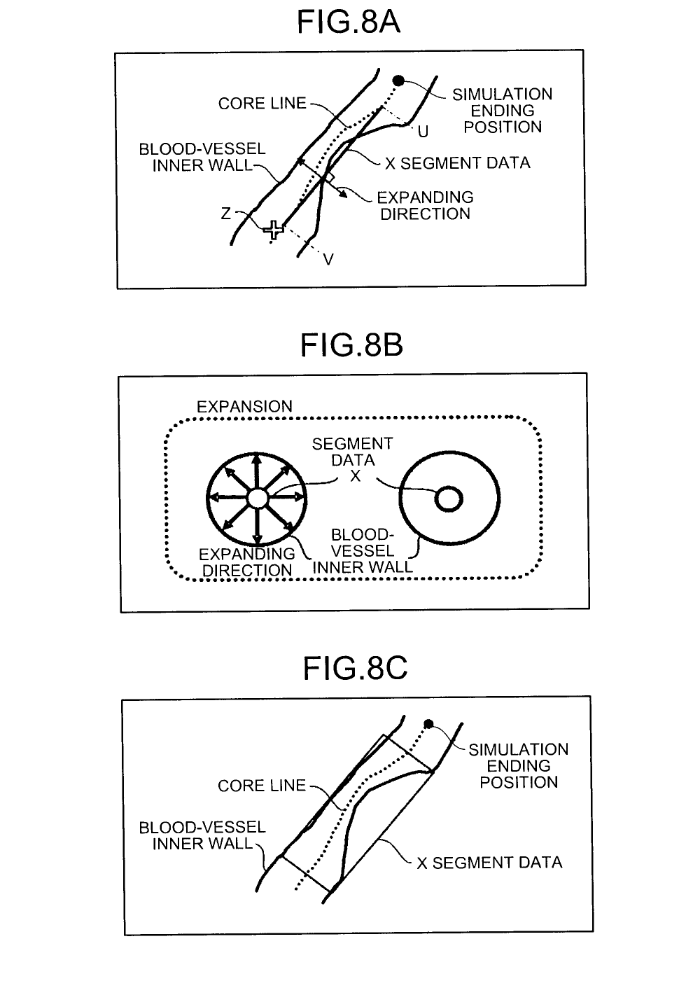

The creation of the segment data X is performed as shown in FIG. 8A. In other words, the length of segment data to be created corresponds to the stent length set in the item e at Step S4. The position at which the segment data is to be created is on a straight line between two points on the core line.

As shown in FIG. 8A, the two points on the core line are a point U a few centimeters short of the simulation ending position, and a point V on the core line at a distance of the length set in the item e from the point U. The distance between the points U and V is the stent length specified by the user. The segment data X is created on the straight line between the points U and V. As a result, the segment data X that is created is assumed as the stent before expansion, and it is assumed that the stent is made to stay.

It is then assumed that a lesion portion (stenotic portion) is to be treated by expanding the stent, the segment data X that is created is expanded based on an expansion request from the user as shown in FIG. 8B. The segment data X is expanded in a direction perpendicular to the straight line between two of the points U and V that are used when creating the segment data.

As a result, as shown in FIG. 8C, the segment data X is displayed substantially as thick as a blood vessel diameter is, and displayed as like the lesion portion (stenotic portion) is treated by expanding the stent.

The input information is assumed that an expansion request is to be input via the GUI, but not limited to the GUI, can also be input by clicking a button of the mouse 821.

The distance from the simulation ending position to the point U, and CT values and an expansion rate of the segment data X can be arbitrarily changed by setting an environment of the simulation. Particularly, because CT values depend on conditions of collecting images, specific numerical values of the CT values are not described, still the CT values need to be set higher than CT values of the coronary artery, and opacity for the CT values needs to be opaque, because each embodiment according to the present invention is configured to be implemented on an extracted coronary artery.

A setting method for a simulation range (starting/ending) is explained below. Although the parameters required in a simulation are described at Step S4 shown in FIG. 5, the setting method for a simulation range (starting/ending) is explained below in detail.

When a specified coronary artery (LCA or RCA) is extracted from a 3D volume image, a starting/ending-position overlay is displayed on the 3D volume image. As shown in FIG. 8A, the starting/ending-position overlay is displayed with the core line (dotted line) of the coronary artery, and a cursor Z (symbol +).

When setting a simulation range, the user drags the cursor Z displayed on the 3D image (FIG. 8A) with the mouse 821, and moves the cursor Z to a position from which the simulation is to be started. The user then selects a start button shown in FIG. 6A, so that the position at which the cursor Z is displayed is set as a simulation starting position.

Similarly, the user drags the cursor Z with the mouse, and moves to a position at which the simulation is to be finished, and then selects an end button in FIG. 6A. As a result, the position at which the cursor Z is displayed is set as a simulation ending position. When setting of the starting and ending positions is completed, the cursor Z is turned not to be displayed. In addition, the set starting/ending positions can be discarded by selecting a clear button shown in FIG. 6A.

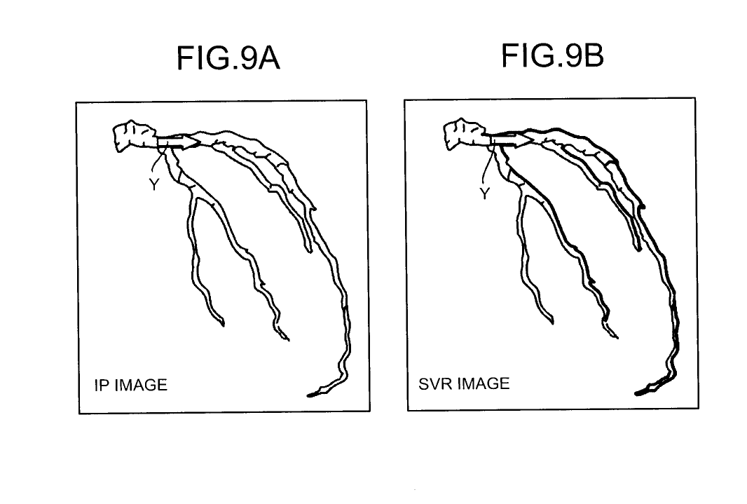

An overlay display of the catheter position is explained below. When setting of the starting and ending positions is completed, as shown in FIGS. 9A and 9B, an overlay image Y that indicates a position and a direction of the catheter is displayed on a 3D projection image and a 3D volume image (SVR). FIG. 9A is the 3D projection image, and FIG. 9B is an SVR image that is the 3D volume image.

The overlay image Y that is displayed is assumed as the catheter tip that is working in examination/treatment, and is to be displayed by renewing a display position/direction each time of input of information based on the input information from the user. When the catheter position has reached the simulation ending position (lesion portion), an image of the segment data X (FIG. 8C) is displayed instead of displaying the marker (arrow), as the overlay image Y.

As a display example of the overlay image Y, the marker in an arrow shape that indicates a position and a direction of the catheter is suitable, however, any shape can be used as long as the shape can indicate the position and the direction of the catheter.

A display direction of the 3D volume image is explained below. According to the embodiments of the present invention, a plurality of display angle patterns are prepared in advance when displaying a 3D volume image, so that a 3D image viewed from an arbitrary angle can be displayed. The display angle patterns mean 3D images of a subject viewed from a plurality of angle directions, so that 3D images of a coronary artery viewed from the angle directions can be acquired as the X-ray CT apparatus 100 performs reconstruction processing.

The display angles are the same as general irradiation angles when the X-ray circulatory-diagnosis apparatus 200 images coronary arteries, and information required for replicating an actual catheter examination/treatment can be provided.

The right screen in FIG. 6A depicts the GUI when specifying a display angle pattern, and numbers 1 to 8 in boxes denote directions of imaging the subject when the coronary artery (LCA) is specified. For example, when the user points a cursor onto the number 1, the user can select a 3D image when irradiating an X-ray from the front of the subject.

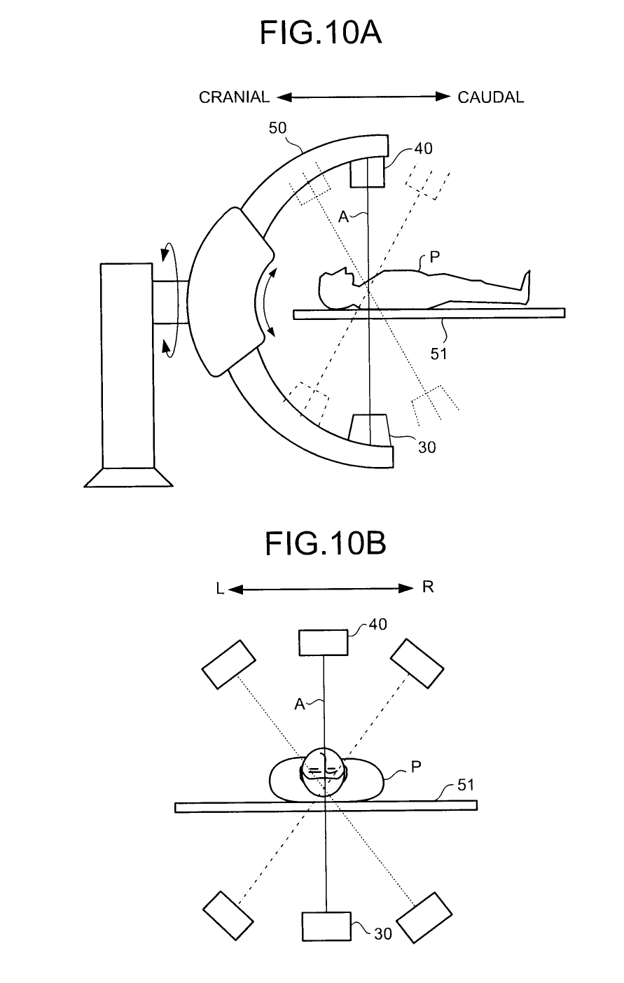

FIGS. 10A and 10B are schematic diagrams for briefly explaining irradiation directions of X-rays in the X-ray circulatory-diagnosis apparatus 200. FIG. 10A is a schematic diagram illustrating the X-ray circulatory-diagnosis apparatus 200 viewed from a lateral side of a subject, and FIG. 10B is a schematic diagram viewed from the head side of the subject.

According to FIGS. 10A and 10B, the arm 50 is attached with the X-ray generating unit 30 and the X-ray detecting unit 40, the X-ray generating unit 30 and the X-ray detecting unit 40 are arranged on opposite sides of the tabletop 51, and the arm 50 can be rotated toward a Cranial (head) direction and a Caudal (leg) direction of the subject as shown in FIG. 10A. Moreover, as shown in FIG. 10B, the arm 50 can be rotated leftward and rightward (L-R) around the subject.

For example, assuming that the X-ray detecting unit 40 is in a reference position when the X-ray detecting unit 40 is positioned in the middle of the Cranial direction and the Caudal direction, and in the middle of L-R, the position on a reference line A corresponds the point of the number 1 in FIG. 6A. The number 2 in FIG. 6A indicates a display angle when the arm 50 is rotated to the right (R) by 30 degrees with respect to the reference line A.

Similarly, the number 3 indicates a display angle when the arm 50 is rotated to the right (R) by 30 degrees and to the Caudal direction by 30 degrees, the number 4 indicates a display angle when the arm 50 is rotated to the Caudal direction by 30 degrees, and the number 5 indicates a display angle when the arm 50 is rotated to the Cranial direction by 35 to 40 degrees. The other numbers also indicate display angles when the arm 50 is rotated in the L-R direction and the Cranial-Caudal direction by predetermined degrees.

The right screen in FIG. 6B depicts directions of imaging the subject when the coronary artery (RCA) is specified. The number 9 in FIG. 6B indicates a display angle when the arm 50 is rotated to the left (L) by 50 to 60 degrees with respect to the reference line A, the number 10 indicates a display angle when the arm 50 is rotated to the Cranial direction by 30 to 40 degrees, and the number 11 indicates a display angle when the arm 50 is rotated to the right (R) direction by 30 degrees. Accordingly, by selecting a display angle (the numbers 1 to 11) on the right screen in FIG. 6A or 6B, a 3D image viewed from an angle desired by the user can be displayed.

Moreover, by selecting a rotation button in FIG. 6A or 6B, a 3D image can be displayed at a display angle further rotated by +90° from each of the display angle patterns. In such case, while the rotation button is being selected, one of the number buttons is selected, so that after a 3D volume image is rotated to an angle corresponding to the selected number, the 3D volume image can be displayed at an angle further rotated, for example, by +90° with respect to the x axis of the screen coordinate system. Accordingly, an overlap state of a coronary artery in the depth direction, which cannot be obtained from a perspective image during an examination/treatment, can be displayed.

A display angle of each of the 3D images shown in FIGS. 9A and 9B can be specified with each of the GUIs shown on the right screens in FIGS. 6A and 6B, and a display angle available to be specified varies depending on a coronary artery (LCA or RCA) to be simulated. The image shown in FIG. 9A or 9B, and the GUI shown in FIG. 6A or 6B are displayed on the same screen as an actual display screen, so that a display angle can be easily specified.

Thus, according to the first embodiment of the present invention, as the image viewer terminal 400 includes the medical-diagnosis assisting apparatus 70 shown in FIG. 4, a user can display a 3D image and can perform a simulation of inserting a catheter prior to an examination/treatment by using image data imaged by the X-ray CT apparatus 100 in advance.

When the X-ray CT apparatus 100 includes the configuration shown in FIG. 4, a radiodiagnosis apparatus that has a simulation function can be provided. In such case, the medical-diagnosis assisting apparatus 70 shown in FIG. 4 can be incorporated inside the image-data processing unit 26 shown in FIG. 2, and the data storage unit 24, the input unit 23, and the display unit 27 shown in FIG. 2 can be used as the storage device 81, the input unit 82, and the display unit 83, respectively.

### Second Embodiment

A second embodiment according to the present invention relates to a radiodiagnosis apparatus that includes a guide function during a catheter examination/treatment, which uses part of the simulation function described above.

In other words, when the X-ray circulatory-diagnosis apparatus 200 includes the medical-diagnosis assisting apparatus 70 shown in FIG. 4, a radiodiagnosis apparatus that has a guide function is provided. In such case, the medical-diagnosis assisting apparatus 70 and the storage device 81 shown in FIG. 4 can be incorporated inside the image-data generating-storing unit 52 shown in FIG. 3, and the input unit 53 and the monitor 55 can be used as the input unit 82 and the display unit 83, respectively.

According to the second embodiment, the X-ray circulatory-diagnosis apparatus 200 can display an actual perspective image acquired by itself, reads three-dimensional image data that were collected in advance and reconstructed by the X-ray CT apparatus 100, and can display a three-dimensional image of a coronary artery at an arbitrary display angle based on CT values of the read image data. The three-dimensional image of the coronary artery serves as a guide image when inserting a catheter.

According to the second embodiment, the coronary-artery extracting unit 71 and the image displaying-direction setting unit 78 shown in FIG. 4 are mainly used. The coronary-artery extracting unit 71 reads three-dimensional image data that were collected/reconstructed by the X-ray CT apparatus 100 and stored in the storage device, extracts a coronary artery (LCA: left coronary artery, RCA: right coronary artery) based on CT values of the read image data, and displays a three-dimensional image (for example, SVR: Shaded Volume Rendering) of the coronary artery onto the monitor 55.

The image displaying-direction setting unit 78 sets for displaying the three-dimensional image of the coronary artery at a display angle specified by a user. The three-dimensional image of the coronary artery is displayed on the display unit 83 together with a perspective image acquired by the X-ray circulatory-diagnosis apparatus 200.

Accordingly, the user can perform the catheter examination/treatment while watching the three-dimensional image of the coronary artery and arbitrarily changing the display angle of the three-dimensional image. Additionally, an actual perspective image is displayed, so that the catheter examination/treatment can be assisted.

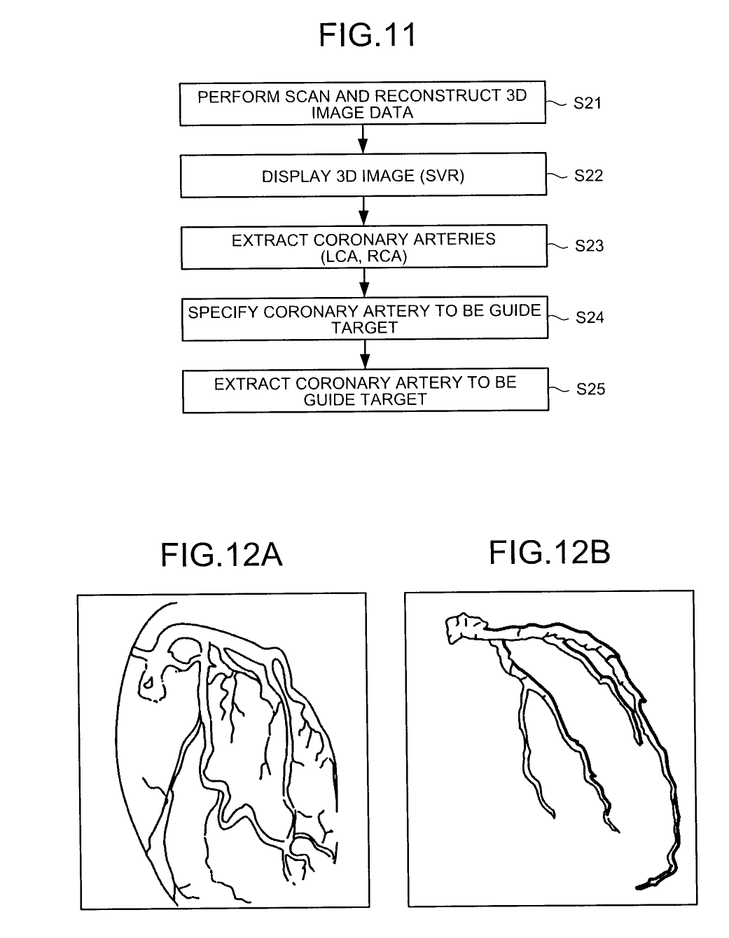

FIG. 11 is a flowchart for explaining operation the X-ray circulatory-diagnosis apparatus 200 according to the second embodiment of the present invention. First of all, at Step S21 in FIG. 11, to collect contrasted blood-vessel images of a subject, the X-ray CT apparatus 100 performs a scan with administration of a contrast medium, and reconstructs three-dimensional image data of the subject. The reconstructed three-dimensional image data are stored in the storage device 81.

Then, at Step S22, a 3D volume image is displayed based on the three-dimensional image data stored in the storage device 81. As the 3D volume image, a shaded volume rendering image (SVR: Shaded Volume Rendering) is displayed.

The displayed 3D volume image includes contrasted blood vessels and regions to be deleted for the simulation. For this reason, at Step S23, only a contrasted blood-vessel region (coronary arteries (LCA: left coronary artery, RCA: right coronary artery)) is extracted from the 3D volume image based on CT values of the image data. As a coronary-artery extraction algorithm, for example, the method described in the aforementioned document 1 is used.

Then, parameters to be required for performing the guide function are set at Step S24. A coronary artery (LCA or RCA) to be a guide target is specified at this step. To specify a coronary artery (LCA or RCA), the user selects one of the items LCA or RCA in the GUI (on the left screen) in FIG. 6A or 6B.

Finally at Step S25, as the coronary artery to be a guide target is specified at Step S24, only the specified coronary artery (LCA or RCA) is extracted from the 3D volume image, and the extracted coronary artery is displayed as a guide image during the catheter examination/treatment.

By using the guide function, a display direction of the 3D volume image can be set. Setting of a display direction is performed by using the GUI (on the right screen) shown in FIG. 6A or 6B similarly to the setting of a display angle of a 3D volume image during a simulation (explanation of this is omitted).

In this way, the display unit 83 displays thereon a perspective image shown in FIG. 12A acquired by the X-ray circulatory-diagnosis apparatus 200, and a three-dimensional image of a coronary artery shown in FIG. 12B.

Additionally, a portion specified as a lesion portion (a stenotic portion) (equivalent to the segment data shown in FIG. 7C) can be displayed on the 3D volume image during the guide function as an overlay image. Accordingly, a position of the lesion portion is specified in advance, so that a course through which a catheter is to be inserted can be grasped more easily.

Furthermore, the overlay image does not need to be displayed constantly, so that the X-ray circulatory-diagnosis apparatus 200 can be configured to switch display/non-display of the overlay image based on input information from the user, and it is adequate to display an overlay with a different shape as long as the user can grasp the lesion portion, and the amount of information provided to the user as a guide function is not substantially reduced.

### Third Embodiment

A simulation by using a still image is explained above in the first embodiment. However, actual catheter operation is carried out while watching a perspective image in motion, because a heart or other parts is moving. Therefore, by using a moving image rather than a still image during a simulation, a more realistic image can be provided.

During catheter operation, a contrast medium is injected to make blood vessels more visible. Therefore, by simulating change in a blood-vessel image due to an injection of a contrast medium, a more realistic image can be provided. During a simulation, an MIP image created by assuming an X-ray perspective image during catheter operation is displayed. Accordingly, by simulating change in the blood-vessel image due to the injection of the contrast medium also for the MIP image, a more realistic image can be provided.

A medical-diagnosis assisting apparatus that can provide a more realistic image by using a moving image and simulating an injection of a contrast medium is explained below as a third embodiment of the present invention.

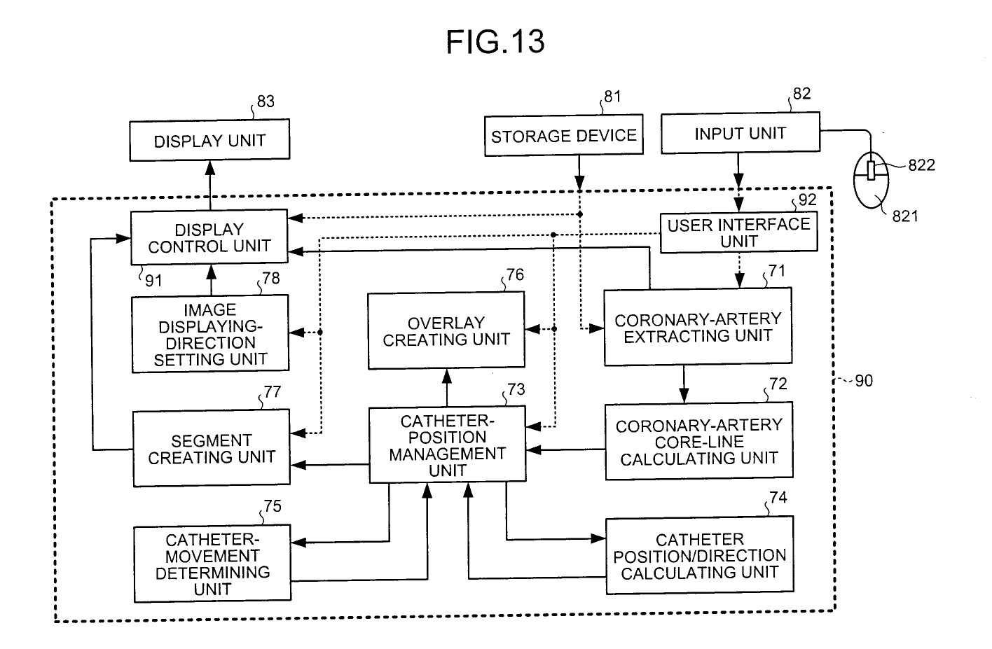

To begin with, a configuration of the medical-diagnosis assisting apparatus according to the third embodiment is explained below. FIG. 13 is a block diagram illustrating a configuration of a medical-diagnosis assisting apparatus 90 according to the third embodiment. For convenience of explanation, functional units that perform functions similar to those performed by the units shown in FIG. 4 are assigned with the same reference numerals, and detailed explanations of them are omitted.

The medical-diagnosis assisting apparatus 90 displays SVR images in a plurality of time phases in cine mode. Therefore, each of the functional units of the medical-diagnosis assisting apparatus 90 functions by dealing with a plurality of time phases of image data. For example, the storage device 81 stores therein a plurality of time phases of image data, and the coronary-artery extracting unit 71 extracts a coronary artery in each of the time phases.

As shown in FIG. 13, the medical-diagnosis assisting apparatus 90 further includes a display control unit 91 and a user interface unit 92, compared with the medical-diagnosis assisting apparatus 70 shown in FIG. 4.

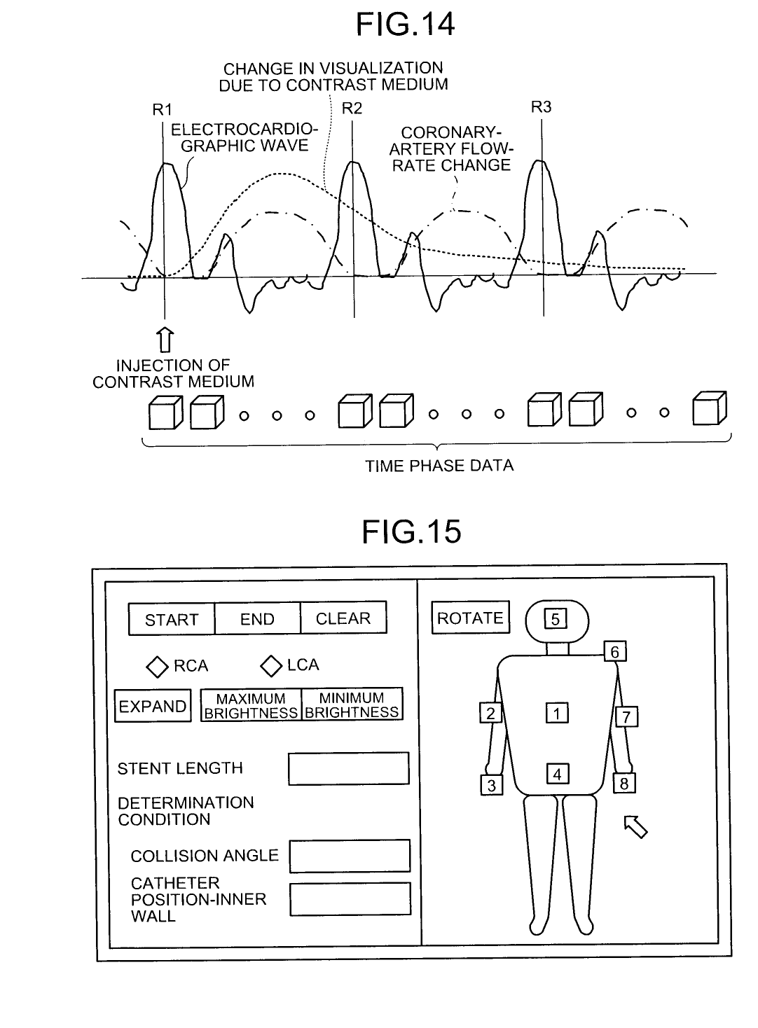

The display control unit 91 repeatedly displays in cine mode (movie display) coronary-artery images in a plurality of time phases extracted from image data by the coronary-artery extracting unit 71 onto the display unit 83. Image data that presents change in visualization due to an injection of a dose of a contrast medium are used as image data in the time phases. FIG. 14 is a schematic diagram that depicts change in visualization due to an injection of a dose of a contrast medium correspondingly to an electrocardiographic wave and coronary-artery flow-rate change. The display control unit 91 repeatedly displays a moving image corresponding to the change in visualization shown in FIG. 14.

Specifically, the display control unit 91 sets opacity (0.0 to 1.0) that is one of imaging conditions of SVR images with respect to each of the time phases correspondingly to the change in visualization shown in FIG. 14, and extracts and displays only a coronary artery from a current catheter position to peripheral blood vessels in each of the time phases. During operation, when the contrast medium is injected with timing R1, the coronary artery is briskly visualized between R1 and R2 due to a flow rate of the coronary artery, and after approximately three heartbeats, the coronary artery is not visualized at all. Such visualization change due to the contrast medium is applied to setting of opacity. For example, suppose the number of the phases is 30 (the number of time phases between R-R is 10), an opacity at the injection of the contrast medium (first time phase) is set to the minimum value (0.0), an opacity around the seventh time phase is set to the maximum value (1.0), and an opacity at the last time phase is set to the minimum value (0.0).

Thus, the display control unit 91 sets opacity at each of the time phases, and extracts only the coronary artery from the current catheter position to peripheral blood vessels in each of the time phases and makes cine display, thereby displaying images that reflect the motion of the heart and a state of the contrast-medium injection from the catheter.

Although the maximum value is set to 1.0 in the example, the maximum value 1.0 is not a fixed value, and can be changed arbitrarily. A contrast medium is often injected bit by bit during catheter operation, and opacity often does not reach the maximum value in some cases. As the maximum value is variable, images that more effectively reflect a state of a contrast-medium injection can be displayed.

When displaying MIP images assuming an X-ray perspective image during operation, the display control unit 91 makes cine display by changing the brightness of each piece of image data in each of the time phases. Specifically, the user sets brightness through the following procedure, and the display control unit 91 sets brightness of all of the time phases based on the brightness (maximum value/minimum value) set by the user.
(1) Display an image in the first time phase, and set brightness (minimum value).
(2) Select a minimum brightness button on a parameter setting GUI shown in FIG. 15.
(3) Display image data around the seventh time phase, and set brightness (maximum value).
(4) Select a maximum brightness button on the parameter setting GUI shown in FIG. 15.

The display control unit 91 displays an overlay created by the overlay creating unit 76, and a segment created by the segment creating unit 77 onto the display unit 83. Furthermore, the display control unit 91 switches a display angle of the coronary artery based on an instruction from the image displaying-direction setting unit 78.

The user interface unit 92 sets parameters for a simulation based on an instruction from the user. For example, the user interface unit 92 sets a length of the stent, and determination conditions.

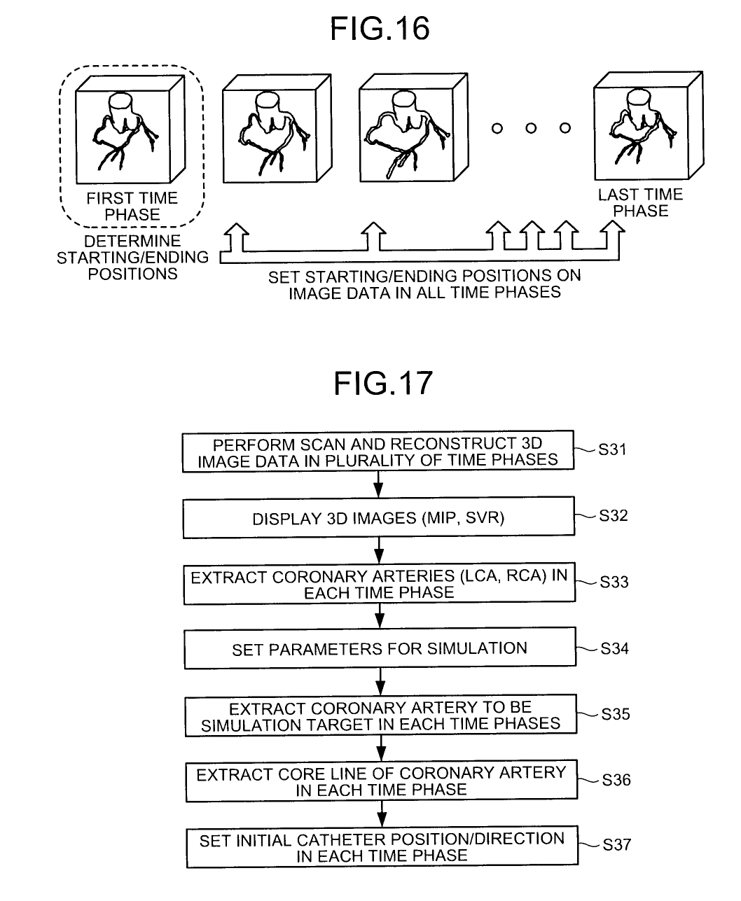

Moreover, the user interface unit 92 sets starting/ending positions of the simulation on images in all of the time phases, as shown in FIG. 16. Specifically, the user sets starting/ending positions on an image in the first time phase by the method explained in the first embodiment. The user interface unit 92 then identifies a blood vessel on which the starting/ending positions are set in the coronary artery based on information about the starting/ending positions set on the image data in the first time phase, and calculates a distance from the starting position to the ending position, thereby setting starting/ending positions on image data in all of the time phases. The identification of the blood vessel of the coronary artery on which the starting/ending positions are set can be performed by extracting details, such as a left anterior descending coronary artery (LAD) and a left circumflex coronary artery (LCX), when the coronary artery is extracted.

Operation of the medical-diagnosis assisting apparatus 90 is explained below with reference to a flowchart in FIG. 17. FIG. 17 presents a procedure from extracting a coronary artery until staring a simulation of a catheter examination by using the extracted coronary-artery image.

At Step S31 in FIG. 17, first of all, to make cine display, the X-ray CT apparatus 100 executes a scan with administration of a contrast medium, and reconstructs three-dimensional image data of a subject in a plurality of time phases. The reconstructed three-dimensional image data in the time phases are stored in the storage device 81.

At Step S32, the three-dimensional image data in the first time phase stored in the storage device 81 are read, and three-dimensional images (a 3D projection image and a 3D volume image) in the first time phase are displayed on the display unit 83. Then at Step S33, coronary arteries that are a contrasted blood-vessel region are extracted from the 3D volume images in each of the time phases based on CT values of the image data.

At Step S34, parameters to be required for performing a simulation, i.e., a coronary artery (LCA or RCA) to be simulated, starting/ending positions, determination conditions, a stent length, and the like are set. When setting starting/ending positions, the user sets only starting/ending positions in the first time phase, and those in the other time phases are automatically set by the medical-diagnosis assisting apparatus 90.

Then at Step S35, the coronary artery (LCA or RCA) specified at Step S34 as a simulation target is extracted in each of the time phases. At Step S36, a core line of the extracted coronary artery is extracted in each of the time phases within the simulation range specified at Step S34. At Step S37, initial catheter position and direction are set in each of the time phases based on the starting position in each of the time phases. The medical-diagnosis assisting apparatus 90 then starts cine display, and turns into a stand-by state of waiting a new input (a renewal request for the catheter position/direction) from the user.

As described above, according to the third embodiment, the medical-diagnosis assisting apparatus 90 is configured to make cine display of three-dimensional images of a coronary artery by using image data in a plurality of time phases, thereby providing a simulation environment with a more realistic image. Moreover, the medical-diagnosis assisting apparatus 90 is configured to indicate change in visualization due to a contrast medium by using opacity of an SVR image and brightness of an MIP image, and to visualize only a coronary artery from the position of a catheter to peripheral blood vessels, thereby providing a simulation environment with a further realistic image. The medical-diagnosis assisting apparatus 90 can be configured to provide a user with a sense more similar to an actual operation by displaying a perspective projection of three-dimensional image display of an SVR image, an MIP image, and the like.

As described above, according to the embodiments of the present invention, information required for an examination (treatment) can be sufficiently obtained prior to a catheter examination, and an examination/treatment can be assisted by watching a simulation image or a guide image, so that safety can be improved. Moreover, shortening of examination/treatment time and reduction of load onto the subject can be achieved.

Although extraction of an image of a coronary artery is explained as an example in the above explanations, an image of other blood vessels can be extracted and displayed as a simulation image or a guide image.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical-diagnosis assisting apparatus comprising:
an extracting unit that extracts image data of a blood vessel portion to be an observation target from three-dimensional image data acquired by imaging a subject;
a display unit that can display a rendering image of the blood vessel portion extracted by the extracting unit;
a display-direction setting unit that displays the rendering image of the extracted blood vessel portion on the display unit at a display angle specified by a user; and
a simulation-image creating unit that simulates a course of a catheter when the catheter is inserted into the blood vessel portion extracted by the extracting unit, and overlays a maker indicating a position and a moving direction of the catheter on the rendering image of the blood vessel portion.

2. The apparatus according to claim 1, wherein the display angle is configured to correspond to an imaging angle when the subject is imaged by an X-ray diagnostic apparatus.

3. The apparatus according to claim 1, wherein the rendering image of the blood vessel portion displayed on the display unit is any one of an X-ray projection image and a 3D volume image.

4. The apparatus according to claim 2, wherein the rendering image of the blood vessel portion displayed on the display unit is any one of an X-ray projection image and a 3D volume image.

5. The apparatus according to claim 1, wherein
the three-dimensional image data acquired by imaging the subject includes data that are reconstructed from image data collected by any one of an X-ray CT apparatus and a magnetic-resonance diagnostic apparatus, and includes rendering images of the subject viewed from a plurality of different angle directions, and
the display-direction setting unit selects the rendering image of the blood vessel portion at the display angle specified by the user, and supplies the selected rendering image to the display unit.

6. The apparatus according to claim 5, wherein the different angle directions are configured to correspond to imaging angles when the subject is imaged with an X-ray diagnostic apparatus from a plurality of imaging directions.

7. The apparatus according to claim 1, wherein the simulation-image creating unit includes
a core-line calculating unit that calculates a core line of a blood vessel extracted by the extracting unit,
a catheter position/direction calculating unit that calculates a position and a direction of a catheter inside the extracted blood vessel,
a catheter-movement determining unit that determines an acceptability of a move of the catheter in accordance with a state of the blood vessel portion,
a catheter-position management unit that determines a position of the catheter based on core-line information obtained by the core-line calculating unit and input information from a user in cooperation with the catheter position/direction calculating unit and the catheter-movement determining unit, and
an overlay creating unit that creates maker information indicating a position and a moving direction of the catheter based on the information obtained by the catheter-position management unit.

8. The apparatus according to claim 7, wherein the simulation-image creating unit allows a user to set at least a distance between a tip of the catheter and an inner wall of the blood vessel, and a collision angle between the tip of the catheter and the inner wall of the blood vessel by using a graphical user interface through which parameters required for performing a simulation are set, and determines an acceptability of a move of the catheter determined based on the set parameters.

9. The apparatus according to claim 7, further comprising a segment creating unit that creates segment data with which a stent is displayed when the catheter has reached a lesion portion in the blood vessel portion based on information obtained by the catheter-position management unit and input information from the user, wherein an image based on the segment data is displayed instead of the marker in an overlaid manner on the rendering image of the blood vessel portion.

10. The apparatus according to claim 9, wherein the segment creating unit allows the user to set a length of the stent based on input information from the user.

11. The apparatus according to claim 1, wherein
the extracting unit extracts respective image data of a blood vessel portion to be an observation target from three-dimensional image data in a plurality of time phases,
the display unit displays rendering images in a plurality of time phases as a moving image, and
the simulation-image creating unit overlays the maker on each of the rendering images in the time phases.

12. The apparatus according to claim 11, further comprising:
a contrast-medium simulation-image creating unit that creates rendering images that simulate movement of a contrast medium to be injected from the catheter based on the image data extracted by the extracting unit and a course of the catheter simulated by the simulation-image creating unit, wherein
the display unit displays the rendering images created by the contrast-medium simulation-image creating unit as a moving image.

13. A radiodiagnosis apparatus comprising:
a projection-image data creating unit that creates perspective image data of a subject with an X-ray diagnostic apparatus;
an extracting unit that acquires three-dimensional image data obtained by imaging the subject with any one of an X-ray CT apparatus and a magnetic-resonance diagnostic apparatus, and extracts image data of a blood vessel portion to be an observation target;
a display-direction setting unit that sets a display angle of a rendering image of the blood vessel portion extracted by the extracting unit based on a specification specified by a user; and
a display unit that can display a perspective image of the blood vessel portion created by the projection-image data creating unit, and a rendering image of the blood vessel portion at the display angle specified by the user.

14. The apparatus according to claim 13, wherein the rendering image of the blood vessel portion is displayed as a guide image when performing any one of an examination and a treatment by inserting a catheter into a blood vessel portion of the subject.

15. A medical-diagnosis assisting method comprising:
extracting image data of a blood vessel portion to be an observation target from three-dimensional image data acquired by imaging a subject;
creating a maker that indicates a position and a moving direction of a catheter by simulating a course of the catheter when the catheter is inserted into the extracted blood vessel portion;
displaying a three-dimensional image of the extracted blood vessel portion on a display unit at a display angle specified by a user; and
displaying the maker by overlaying on the three-dimensional image of the blood vessel portion.
